(19) United States
(12) Patent Application Publication
(10) Pub. No.: US 2025/0049709 A1
(43) Pub. Date: Feb. 13, 2025

(54) ZIPRASIDONE FORMULATIONS

(71) Applicant: BELOTECA, INC., San Diego, CA (US)

(72) Inventors: Frederik Defesche, San Diego, CA (US); Dorla Mirejovsky, San Diego, CA (US); Cristiane Marcondes Messora Barion, San Diego, CA (US)

(73) Assignee: BELOTECA, INC., San Diego, CA (US)

(21) Appl. No.: 18/717,958

(22) PCT Filed: Dec. 9, 2022
(86) PCT No.: PCT/US2022/081320

§ 371 Date: Jun. 7, 2024

Related U.S. Application Data
(60) Provisional application No. 63/288,537, filed on Dec. 11, 2021.

(51) Int. Cl. A61K 9/08; A61K 31/496; A61K 47/20; A61K 47/40

(52) U.S. Cl. CPC A61K 9/08; A61K 31/496; A61K 47/20; A61K 47/40

(57) ABSTRACT

Provided is an aqueous formulation of ziprasidone, stored for up to six months at 40° C., suitable for intramuscular injection. In these embodiments, the aqueous formulation (a) provides greater than 98% purity of the ziprasidone and/or (b) has less than 0.5% of USP Related Compound C with respect to ziprasidone. Also provided is a method of administering ziprasidone to a patient, the method comprising administering the above formulation to the patient intramuscularly through a syringe.

### CROSS-REFERENCE TO RELATED APPLICATIONS

This application claims the benefit of U.S. Provisional Application No. 63/288,537, filed Dec. 11, 2021, and incorporated by reference herein in its entirety.

### BACKGROUND OF THE INVENTION

### (1) Field of the Invention

The present application generally relates to pharmaceutical formulations. More specifically, aqueous formulations of ziprasidone are provided that are stable for long periods of time.

### (2) Description of the Related Art

GEODON® for Injection, active ingredient ziprasidone mesylate trihydrate, is supplied as a lyophilized product for intramuscular injection. GEODON® for Injection is available in a single dose vial as ziprasidone mesylate (20 mg ziprasidone/mL when reconstituted according to label with 1.2 mL sterile water for injection). Each mL of ziprasidone for injection after reconstitution contains 20 mg of ziprasidone and 4.7 mg of methanesulfonic acid solubilized by 294 mg of sulfobutyl ether β-cyclodextrin sodium (SBECD). Ziprasidone is indicated for the treatment of acute manic or mixed episodes associated with bipolar disorder, with or without psychotic features.

Because the reconstitution procedure is inconvenient and subject to error, an aqueous formulation of ziprasidone mesylate trihydrate would be useful. The present invention provides such an aqueous formulation of ziprasidone mesylate trihydrate.

### BRIEF SUMMARY OF THE INVENTION

The present invention contemplates an aqueous formulation that contains a psychotropic effective amount ziprasidone or a pharmaceutically acceptable salt and/or hydrate thereof, that is storage-stable for up to six months at 40° C., and suitable for intramuscular injection. In these embodiments, the aqueous formulation
(a) provides greater than 98% purity of the ziprasidone, its pharmaceutically acceptable salt and/or hydrate and/or
(b) has less than 0.5% of USP Related Compound C with respect to ziprasidone itself.

Also provided is a method of administering ziprasidone to a patient, the method comprising administering the above formulation to the patient intramuscularly through a syringe.

### DETAILED DESCRIPTION OF THE INVENTION

The present invention is based in part on the discovery, as described in the Examples below, that ziprasidone mesylate trihydrate, as present in GEODON® for Injection, is surprisingly stable in aqueous solution, e.g., the solution present when lyophilized GEODON® for Injection is reconstituted with water according to the label instructions, provided that oxygen exposure to the reconstituted ziprasidone is limited, e.g., by storage in a sealed vessel (e.g., a vial or a syringe) with minimal headspace; i.e., the unfilled space left above the contents in a sealed container, and/or with N₂, Ar or another pharmaceutically acceptable gas that is not reactive with the reconstituted composition constituents, replacing the atmosphere in the headspace, and/or the vessel packaged in oxygen limiting packaging, e.g., with an oxygen scavenger, for example as provided in PCT Patent Publication WO 2020/257134.

Thus, in some embodiments, an aqueous formulation of ziprasidone or its pharmaceutically acceptable salt is provided. For case of expression, the word “ziprasidone” is used hereinafter to include ziprasidone itself and also a pharmaceutically acceptable salt thereof, unless the word is used with another word such as “alone”, “itself” or in a context such as in the preparation of a pharmaceutically acceptable salt that indicates that the compound is in free base form.

The formulation, after storage for an extended period of time, e.g., up to six months at 40° C., or twelve months at 2-25° C., is suitable for intramuscular injection. As used herein, “suitable for intramuscular injection” is a ziprasidone formulation that provides greater than 98% purity of the ziprasidone itself and/or has less than 0.5% of USP Related Compound C with respect to ziprasidone, aside from the presence of a neutralizing acid or other formulation ingredient.

As used herein, “USP Related Compound C” is a ziprasidone dimer, having an ultra-performance liquid chromatography (UPLC) relative retention time (RRT) of 1.73-1.75 under conditions described in Example 6.

The above-described extended storage of aqueous ziprasidone can be achieved by limiting oxygen exposure of the formulation, as further described below and in the examples. The ziprasidone, its pharmaceutically acceptable salt and/or hydrate in these formulations can be in any form including any hydration (e.g., anhydrous, monohydrate, dihydrate or trihydrate), and any pharmaceutically acceptable salt form (including but not limited to mesylate or hydrochloride). In some embodiments, the ziprasidone is ziprasidone mesylate trihydrate.

A contemplated compound, ziprasidone, is an amine and can typically be used in the form of a pharmaceutically acceptable acid addition salt derived from an inorganic or organic acid. Exemplary salts include but are not limited to the following: acetate, adipate, alginate, citrate, aspartate, benzoate, benzenesulfonate, bisulfate, butyrate, camphorate, camphorsulfonate, digluconate, cyclopentanepropionate, dodecylsulfate, ethanesulfonate, glucoheptanoate, glycerophosphate, hemisulfate, heptanoate, hexanoate, fumarate, hydrochloride, hydrobromide, hydroiodide, 2-hydroxy-ethanesulfonate, lactate, maleate, methanesulfonate, nicotinate, 2-naphthalenesulfonate, oxalate, palmoate, pectinate, persulfate, 3-phenylpropionate, picrate, pivalate, propionate, succinate, tartrate, thiocyanate, tosylate, mesylate and undecanoate.

The reader is directed to Berge, J. Pharm. Sci. 68 (1): 1-19 (1977) for lists of commonly used pharmaceutically acceptable acids and bases that form pharmaceutically acceptable salts with pharmaceutical compounds.

In these embodiments, the formulation can be stored at any temperature or combinations of temperatures for the indicated period. Examples of storage temperatures suitable for the aqueous ziprasidone formulation are 40, 35, 30, 25, 20, 15, 10, 8, 6, 4, or 2° C., or any temperature in between, or any combinations of those temperatures for a period of time of up to about six months at 40° C. up to about twelve months at 2-25° C.

The ziprasidone formulations, under conditions protected from ambient oxygen, can be stored for up to one month, two months, three months, four months, five months, or up to six months, for formulations stored at 40° C., and up to one month, two months, three months, four months, five months, six months, seven months, eight months, nine months, ten months, eleven months, or up to 12 months for formulations stored below 40° C., while still being suitable for intramuscular injection.

In additional embodiments, a formulation that provides a higher concentration of ziprasidone such as 20 mg/mL further comprises B-cyclodextrin sulfobutyl ether sodium (SBECD). For example, a formulation that provides 20 mg/mL typically utilizes about 294 mg SBECD plus 4.7 mg of methanesulfonic acid, as is provided in GEODON® for Injection.

The ziprasidone in these formulations can be at any concentration, e.g., 1, 2, 5, 10, 15, 20, 25, 30, 35, or about 40 mg/mL, or any concentration in between or outside those concentrations, up its solubility in the aqueous formulation. In some embodiments, the ziprasidone is present at about 2 mg/mL or at about 10 mg/mL to about 20 mg/mL. In specific embodiments, the ziprasidone is present at a concentration of about 20 mg/mL. In various embodiments, doses of 10 mg can be administered every two hours; doses of 20 mg can be administered every four hours up to a maximum of 40 mg/day, based on the amount of ziprasidone itself.

It is to be understood that ziprasidone or a pharmaceutically acceptable salt and/or hydrate thereof can have different solubilities. Thus, ziprasidone itself is reported to have solubility in water at 25° C. of 2.13 mg/mL; [pubchem.ncbi.nlm.nih.gov/compound/Ziprasidone]; ziprasidone hydrochloride is reported to have solubility in water of 0.00718 mg/mL [go.drugbank.com]; and ziprasidone mesylate trihydrate is reported to have a water solubility of 0.013 mg/mL [go.drugbank.com].

In further embodiments, the formulation is contained in a reduced oxygen headspace container. Any reduced oxygen headspace container, now known or later discovered may can be used in these embodiments. As used herein, in one aspect of this embodiment, a reduced oxygen headspace container has about 5 mm or less head space above the liquid contents. Such a reduced oxygen headspace container is also referred to herein as having a minimal oxygen headspace. Illustratively, the formulation is in a container with a minimal oxygen headspace (e.g., less than 5, 4, 3, 2 or 1 mm) between the stopper/plunger and the liquid level. In some of these embodiments, the container is a syringe. In another aspect, a reduced oxygen headspace container is a container whose headspace contains nitrogen or argon or another non-oxidizing gas. In various embodiments, the container is filled under a flow of nitrogen, argon or another non-oxidizing gas. In other embodiments, the container is flushed with the gas prior to filling. In additional embodiments, the container is flushed with the gas prior to and during filling. In some of these aspects, the reduced oxygen container is a vial or ampule with an N₂ (nitrogen gas) or argon (Ar) gas in the headspace.

The ziprasidone formulation can be in a container at any convenient volume, e.g., 5, 4, 3, 2, 1, or 0.5 ml, or any volume between or outside those volumes. In some embodiments, the volume is about 0.5 to about 1.5 ml of about 20 mg ziprasidone per ml.

The pH value of the formulation can be any pH that provides suitable stability of the ziprasidone. In some embodiments, the formulation has a pH value of 2.8 to 4.0. In other embodiments, the formulation has a pH value of about 3.0 to about 3.8. In more specific embodiments, the formulation has a pH value of about 3.2 to about 3.5.

In various embodiments, the ziprasidone formulation is in secondary packaging (e.g., in a syringe) that prevents penetration of oxygen to the formulation (e.g., through a plunger of the syringe). Nonlimiting examples of such secondary packaging are provided in PCT Patent Publication WO2020/257134 A1. In some of these embodiments, the secondary packaging is a pouch that comprises an oxygen scavenger. Nonlimiting examples of such oxygen scavengers are AGELESS® ZPT-100MBC (Mitsubishi Gas Chemical America, Inc., New York, N.Y.) and Multisorb Oxygen Scavenger StabilOx® D00-H75 (Multisorb Technologies, Inc., Buffalo, N.Y.).

The present invention is also directed to a method of administering ziprasidone to a patient. The method comprises administering an above formulation to the patient intramuscularly through a syringe.

Preferred embodiments are described in the following examples. Other embodiments within the scope of the claims herein will be apparent to one skilled in the art from consideration of the specification or practice of the invention as disclosed herein. It is intended that the specification, together with the examples, be considered exemplary only, with the scope and spirit of the invention being indicated by the claims, which follow the examples.

### Example 1. Stability of Lyophilized and Bulk Aqueous Solution of Ziprasidone Mesylate Trihydrate

A bulk solution of ziprasidone mesylate trihydrate, as shown in Table 1, was filled in 5 mL/20 mm vials and lyophilized. The lyophilized product was placed at 60° C. for one month and for 6 months at 40° C. and 25° C. under ambient humidity. The stability program also included the accelerated stability testing of GEODON® for Injection.

Ziprasidone and ziprasidone degradation products were determined using ultra-performance liquid chromatography (UPLC) as described in Example 6 below.

TABLE 1
Composition of Ziprasidone Bulk Solution

| Ingredient | Function | Concentration/mg/mL |
|---|---|---|
| Ziprasidone Mesylate | Active Ingredient | 24.7 mg, eq to 20 mg |
| Trihydrate | | |
| β-Cyclodextrin sulfobutyl | Solubilizer | 294 mg |
| ether sodium, NF | | |
| Purified Water | Diluent | Q.S. to 1 mL |

The ziprasidone in the lyophilized form was very stable. Two impurities were formed RRT 1.74-1.75 and RRT 1.83 at levels of about 0.15% and about 0.10%, respectively, after 6 months. The level of degradation was similar at 40° C. and 25° C. Similar degradation profile was found for GEODON® for Injection. At the same time, it was discovered that the remaining bulk solution, that had been filled as about 5 mL in 20 mL vial, closed under nitrogen with screw Teflon® coated cap, and stored in an upright position for about 4.5 months at 2-8° C., showed the same impurities at almost similar levels as those found as in the lyophilized product with a slight increase in RRT 1.74 in comparison to the initial bulk solution (Table 2).

TABLE 2
Re-Test of Bulk Solution after a Storage of 4.5 Months at 2-8° C.

| | Bulk Solution | | | |
|---|---|---|---|---|
| Test | Initial Testing | | 4.5 Months at 2-8° C. | |
| Physical Appearance | Slightly orange/pink | | Clear, slightly pink/ | |
| | clear solution | | orange solution with | |
| | | | no visible particulates | |
| pH value | 3.84 | | 4.01 | |
| Ziprasidone Assay | 103.9 | | 104.0 | |
| (HPLC), | | | | |
| % LC (20 mg/mL) | | | | |
| Ziprasidone Related | RRT | 1.74-0.09 | RRT | 1.73-1.758: 0.18 |
| Substances, % w/w | RRT | 1.825-0.05 | RRT | 1.82-1.83: 0.07 |

| | |
|---|---|
| Total 0.13 | Total 0.25 |

This unexpected discovery led us to postulate that the development of a liquid product is feasible and warrant further investigation. Liquid product is always preferred over the lyophilized form due to a lower manufacturing cost and easier manipulation for the end-user. Similar results are also found after storage for up to 12 months at 2-8° C. and also at 20-25° C.

### Example 2. Ziprasidone Injection-Effect of Oxygen

An additional bulk solution was prepared in sparged water (with what?? nitrogen??) according to the composition shown in Table 1, filtered, and filled under nitrogen, as a 2.2 mL fill. The container closure selected for the study is listed in Table 3. Vials were also filled under ambient atmospheric conditions for comparison.

TABLE 2
Primary Packaging Components for Ziprasidone Injection

| Component | Supplier |
|---|---|
| 5 mL Clear Type I Glass Molded Vial | SGD Pharma |
| 20 mm Coated 4023/50/Grey B2-40 | Westar RS |

To determine the stability of the of the bulk solution ingredients, vials were placed at 40° C. for up to six months and at and 25° C. for up to twelve months. Results are summarized in Table 4 (40° C. under Nitrogen), Table 5 (25° C., under Nitrogen), Table 6 (40° C., 20% Oxygen), and Table 7 (25° C., 20% Oxygen). Stability testing of vials closed under 20% oxygen (ambient air) was discontinued after 3 months due to high level impurities and discoloration.

Ziprasidone and ziprasidone degradation products were determined using ultra-performance liquid chromatography (UPLC) as described in Example 6 below.

TABLE 4
Stability Ziprasidone Injection (Nitrogen Headspace) Held at 40° C. for 6 Months*

| | | Time Points | | | | |
|---|---|---|---|---|---|---|
| | | Time Zero | 1 Month | 2 Months | 3 Months | 6 Months |
| Test | Specifications | 13 Oct. 2020 | 13 Nov. 2020 | 12 Dec. 2020 | 13 Jan. 2021 | 13 Apr. 2021 |
| Physical Appearance | Clear, colorless to a | Clear Solution, | Clear Solution, | Clear Solution, | Clear Solution, | Clear Solution, |
| | slightly pink or yellow | slightly pink/ | like BY3 | like “E” USP | like “E” USP | like “BY1” Ph |
| | brownish solution free | orange (like R6) | | Standard | Standard | Eur Standard |
| | of visible contaminants | | | | | |
| Ziprasidone Assay, % Label | 90.0-110.0% | 100.8 | 101.4 | 101.3 | 102.1 | 98.6 |
| Claim (20 mg/mL) | | | | | | |

Related Substances via HPLC (w/w %)

| | | | | | | |
|---|---|---|---|---|---|---|
| Specified-Identified | USP Related Compound C | 0.09 | 0.10 | 0.12 | 0.17 | 0.23 |
| Degradation Product | RRT 1.73-1.75 | | | | | |
| Any Unidentified | RRT 1.78 | ND | ND | ND | 0.12 | 0.18 |
| Unspecified | RRT 1.82-1.83 | 0.05 | 0.05 | 0.06 | 0.11 | 0.16 |
| Degradation Product | RRT 2.11-2.12 | ND | ND | 0.06 | ND | 0.13 |
| | RRT 2.17 | ND | ND | ND | 0.08 | 0.19 |
| Total Related Substances | Report Results | 0.14 | 0.15 | 0.24 | 0.65 | 1.12 |
| UV Absorbance | Report Results | NT | NT | NT | 0.451 | 0.359 |
| (Read at 420 nm) | | | | | | |
| Viscosity, cP at 25° C. | Report Results | 4.68 | 4.75 | NT | 4.92 | 4.96 |
| pH value | 2.5-6.0 | 3.88 | 3.80 | NT | 3.81 | 3.82 |
| O₂ Headspace, % Oxygen | Report Results | NT | 0.71 | NT | 0.92 | 1.73 |

*RRT = Relative Retention Time; NMT = Not More Than; ND = Not Detected; NT = Not Tested; and LOQ = Limit of Quantitation (0.05%)

TABLE 3
Stability Ziprasidone Injection (Nitrogen Headspace) Held at 25° C. for 12 Months*

| | | Time Points | | | |
|---|---|---|---|---|---|
| | | Time Zero | 3 Months | 6 Months | 12 Months |
| Test | Specifications | 13 Oct. 2020 | 13 Jan. 2021 | 13 Apr. 2021 | 13 Oct. 2021 |
| Physical Appearance | Clear, colorless | Clear Solution, | Clear Solution, | Clear Solution, | Clear solution, |
| | to a slightly pink | slightly pink/ | like “BY2” | like “BY2” | like “BY3” |
| | or yellow brownish | orange | Ph Eur | Ph Eur | Ph Eur |
| | solution free of | (like R6) | Standard | Standard | Standard |
| | visible contaminants | | | | |
| Ziprasidone Assay, % | 90.0-110.0% | 100.8 | 101.8 | 98.1 | 98.0 |
| Label Claim (20 mg/mL) | | | | | |

Related Substances via HPLC (w/w %)

| | | | | | |
|---|---|---|---|---|---|
| Specified-Identified | USP Related Compound C | 0.09 | 0.14 | 0.14 | 0.26 |
| Degradation Product | RRT 1.73-1.75 | | | | |
| Any Unidentified | RRT 1.82-1.83 | 0.05 | 0.05 | 0.13 | 0.10 |
| Unspecified | | | | | |
| Degradation Product | RRT 2.14-2.16 | ND | ND | 0.12 | ND |
| Total Related Substances | Report Results | 0.14 | 0.19 | 0.47 | 0.49 |
| UV Absorbance | Report Results | NT | 0.193 | 0.183 | 0.229 |
| (Read at 420 nm) | | | | | |
| Viscosity, cP, 25° C. | Report Results | 4.68 | 4.99 | 4.81 | Not tested |
| pH value | 2.5-6.0 | 3.88 | 3.70 | 3.79 | 3.76 |
| O₂ Headspace, % Oxygen | Report Results | NT | 0.93 | 1.03 | 1.35 |

*RRT = Relative Retention Time; NMT = Not More Than; ND = Not Detected; NT = Not Tested; and LOQ = Limit of Quantitation (0.05%)

TABLE 4
Stability Ziprasidone Injection (20% Oxygen) Held at 40° C. for 3 Months*

| | | Time Points | | | |
|---|---|---|---|---|---|
| | | Time Zero | 1 Month | 2 Months | 3 Months |
| Test | Specifications | 13 Oct. 2020 | 13 Nov. 2020 | 12 Dec. 2020 | 13 Jan. 2021 |
| Physical Appearance | Clear, colorless to a slightly | Clear Solution, | Clear Solution, | Clear Solution, | Clear Solution, |
| | pink or yellow brownish | slightly pink/orange | like BY2 | like “G” | like “G” |
| | solution free of visible | (like R6) | | USP Standard | USP Standard |
| | contaminants | | | | |
| Ziprasidone Assay, | 90.0-110.0% | 100.8 | 98.8 | 93.7 | 91.4 |
| % Label Claim | | | | | |
| (20 mg/mL) | | | | | |

Related Substances via HPLC (w/w %)

| | | | | | |
|---|---|---|---|---|---|
| Specified-Identified | USP Related | 0.09 | 1.44 | 1.87 | 2.77 |
| Degradation Product | Compound C | | | | |
| | RRT 1.73-1.75 | | | | |
| Any Unidentified | RRT 0.25 | ND | ND | 0.08 | 0.21 |
| Unspecified | RRT 0.64-0.65 | ND | 0.07 | 0.13 | 0.22 |
| Degradation Product | RRT 0.74 | ND | ND | ND | 0.14 |
| | RRT 0.75 | ND | ND | 0.08 | 0.20 |
| | RRT 1.11 | ND | 0.09 | 0.08 | 0.19 |
| | RRT 1.82-1.83 | 0.05 | 0.74 | 0.98 | 1.59 |
| | RRT 1.88-1.90 | ND | 0.13 | 0.22 | 0.37 |
| | RRT 2.17-2.18 | ND | ND | 0.14 | 0.78 |
| | RRT 2.20-2.23 | ND | ND | 0.08 | 0.32 |
| Total Related Substances | Report Results | 0.14 | 2.76 | 4.07 | 7.29 |
| UV Absorbance | Report Results | NT | NT | NT | 0.979 |
| (Read at 420 nm) | | | | | |
| Viscosity, cP, 25° C. | Report Results | 4.68 | 4.95 | NT | 5.97 |
| pH value | 2.5-6.0 | 3.88 | 3.51 | NT | 3.41 |
| O₂ Headspace, % | Report Results | NT | 18.20 | NT | 17.20 |

*RRT = Relative Retention Time, NMT = Not More Than, ND = Not Detected, NT = Not Tested and LOQ = Limit of Quantitation (0.05%)

TABLE 5
Stability Ziprasidone Injection (20% Oxygen) Held at 25° C. for 3 Months*

| | | Time Points | |
|---|---|---|---|
| | | Time Zero | 3 Months |
| Test | Specifications | 13 Oct. 2020 | 13 Jan. 2021 |
| Physical Appearance | Clear, colorless to a slightly pink | Clear Solution, slightly | Clear Solution, like |
| | or yellow brownish solution | pink/orange (like R6) | “C” USP Standard |
| | free of visible contaminants | | |
| Ziprasidone Assay, % Label Claim | 90.0-110.0% | 100.8 | 97.5 |
| (20 mg/mL) | | | |

Related Substances via HPLC (w/w %)

| | | | |
|---|---|---|---|
| Specified-Identified Degradation | USP Related Compound C | 0.09 | 1.36 |
| Product | RRT 1.73-1.75 | | |
| Any Unidentified Unspecified | RRT 0.54-0.55 | ND | 0.09 |
| Degradation Product: | RRT 0.75 | ND | ND |
| | RRT 0.84 | ND | 0.05 |
| | RRT 1.11 | ND | 0.07 |
| | RRT 1.82-1.83 | 0.05 | 0.51 |
| | RRT 1.88-1.90 | ND | 0.07 |
| Total Related Substances | Report Results | 0.14 | 2.26 |
| UV Absorbance | Report Results | NT | 0.232 |
| (Read at 420 nm) | | | |
| Viscosity, cP at 25° C. | Report Results | 4.68 | 4.87 |
| pH | 2.5-6.0 | 3.88 | 3.52 |
| O₂ Headspace, % | Report Results | NT | 17.98 |

*RRT = Relative Retention Time; NMT = Not More Than; ND = Not Detected; NT = Not Tested; and LOQ = Limit of Quantitation (0.05%)

The stability data indicated:
The development of Ziprasidone Injection—a liquid product—is feasible, provided the liquid product is protected from oxygen.
Ziprasidone in liquid form forms two main degradants: RRT 1.73-1.75, identified as the USP Related Compound C (ziprasidone dimer), and RRT 1.82-1.83. Both degradants are formed under the effect of oxygen. Similar degradants were found in the lyophilized product.
Under nitrogen headspace almost the same levels of these two degradants were found after 6 months regardless of whether the vials were stored at 40° C. or 25° C. The percentage of the Compound C after a 6-month exposure to 40° C. was only slightly higher than the level obtained for a 25° C. exposure (0.23% vs. 0.14%).
Under 20% of oxygen, the formation of Compound C doubled under the effect of heat (1.36% at 3M at 25° C. vs. 2.77% at 40° C.).
The assay value is affected by the oxygen as well as heat.
A discoloration was observed under the effect of oxygen and heat. The discoloration correlated with an increase in the UV absorbance taken at 420 nm.
An increase of various degradants at 6 months indicates that oxygen penetrates through the stopper leading to higher degradation. The oxygen headspace level increased at 40° C. from 0.92% at 3 months to 1.73% at 6 months. The oxygen headspace level increased at 25° C. from 0.93% at 3 months to 1.35% at 12 months.
A decrease of the oxygen level in the headspace in vials filled under ambient conditions indicates that oxygen is consumed by ziprasidone degradation.

### Example 3. Ziprasidone Injection—Feasibility of Product Filled in Syringes

Liquid drug product filled in syringes offers many advantages for convenience of use. Currently, to administer a 10 mg dose, 0.5 mL of the reconstituted solution must be withdrawn from the vial. To administer a 20 mg dose, 1.0 mL of the reconstituted solution is withdrawn. Any unused portion should be discarded.

As ziprasidone Injection is oxygen sensitive it was postulated that filling a product in a syringe with a minimal headspace would eliminate the requirement of filling the vials under reduced oxygen levels.

A new batch of ziprasidone was prepared according to the composition shown in Table 1 and filled as a 1.2 mL-fill in container closure system shown in Table 8.

TABLE 6
Primary Packaging Components for Ziprasidone Injection 20 mg/mL

| Component | Supplier |
|---|---|
| Syringe EZ-Fill 1 mL Long ITC 7025/65 | Ompi |
| Plunger, Article 2340 4023/50 Gray B2-40 | West Pharmaceutical |
| | Services |

Ziprasidone and ziprasidone degradation products were determined using ultra-performance liquid chromatography (UPLC) as described in Example 6 below.

Syringes were placed at 40° C. and 25° C. for 6 months at ambient humidity at horizontal positions. The results for 40° C. conditions are shown in Table 9.

Results for 25° C. samples can be seen in Table 10.

TABLE 7
Stability of Ziprasidone Injection, 20 mg/mL Filled in Syringes, at 40° C.

| 40° C. | Specification | T = 0 | 1 Month | 2 Months | 3 Months | 6 Months |
|---|---|---|---|---|---|---|
| Pull Date | N/A | N/A | 3 Jan. 2021 | 3 Feb. 2021 | 3 Mar. 2021 | 3 Jun. 2021 |
| Appearance | Clear, colorless to a | Clear, slightly | Clear, slightly | Clear solution, | Clear solution, | Clear solution, |
| | slightly pink or yellow | pink/orange solution | pink/orange solution | like “BY1” Ph | like “BY1” Ph | like “B1” Ph |
| | brownish solution free | with no visible | with no visible | Eur Standard | Eur Standard | Eur Standard |
| | of visible contaminants | particulates | particulates | | | |
| Assay (HPLC), % LC | 90.0-110.0% | 100.0% | 101.0% | 99.0% | 100.8% | 96.5% |

Related Substances via HPLC (w/w %)

| | | | | | | |
|---|---|---|---|---|---|---|
| Specified-Identified | USP Related Compound C | 0.07 | 0.40 | 0.64 | 0.47 | 1.20 |
| Degradation Product | RRT 1.72-1.74 | | | | | |
| Any Unidentified | RRT 0.70 | ND | ND | ND | ND | 0.10 |
| Unspecified | RRT 1.82 | 0.06 | 0.17 | 0.29 | 0.25 | 0.66 |
| Degradation Product: | RRT 2.17 | ND | ND | 0.12 | 0.05 | 0.05 |
| | RRT 2.20-2.22 | ND | ND | ND | ND | 0.14 |
| Total Related Substances | Report Results | 0.13 | 0.57 | 1.19 | 0.83 | 2.53 |
| UV Absorbance | Report Results | NT | 0.195 | 0.260 | 0.317 | 0.700 |
| pH value | 2.5-6.0 | 3.71 | 3.59 | 3.49 | 3.52 | 3.44 |
| Test Date | | 3 Dec. 2020 | 4 Jan. 2021 | 4 Feb. 2021 | 2 Mar. 2021 | 7 Jun. 2021 |

ND = Not Detected;
NT = Not Tested;
NA = Not Applicable;
LOQ = Limit of Quantitation, 0.05%;

TABLE 8
Stability of Ziprasidone Injection, 20 mg/mL Filled in Syringes, at 25° C.

| 25° C. | Specification | T = 0 | 1 Month | 3 Months | 6 Months |
|---|---|---|---|---|---|
| Pull Date | N/A | N/A | 3 Jan. 2021 | 3 Mar. 2021 | 3 Jun. 2021 |
| Appearance | Clear, colorless | Clear, slightly pink/ | Clear, slightly pink/ | Clear solution, | Clear solution, no |
| | to a slightly pink or | orange solution | orange solution | like “BY3” Ph | visible particulates, |
| | yellow brownish | with no visible | with no visible | Eur Standard | like “D” Fluka |
| | solution free of visible | particulates | particulates | | Standard |
| | contaminants | | | | |
| Assay (HPLC), % | 90.0-110.0% | 100.0% | 101.0% | 98.7% | 95.7% |
| LC | | | | | |

Related Substances via HPLC (w/w %)

| | | | | | |
|---|---|---|---|---|---|
| Specified-Identified | USP Related | 0.07 | 0.23 | 0.33 | 0.62 |
| Degradation Product | Compound C | | | | |
| | RRT 1.72-1.74 | | | | |
| Any Unidentified | RRT 1.82 | 0.06 | 0.08 | 0.19 | 0.27 |
| Unspecified | | | | | |
| Degradation Product | | | | | |
| Total Related | Report Results | 0.13 | 0.31 | 0.52 | 1.04 |
| Substances | | | | | |
| UV Absorbance | Report Results | NT | 0.089 | 0.138 | 0.194 |
| pH value | 2.5-6.0 | 3.71 | 3.69 | 3.55 | 3.63 |
| Test Date | | 3 Dec. 2020 | 4 Jan. 2021 | 5 Mar. 2021 | 7 Jun. 2021 |

ND = Not Detected; NT = Not Tested; NA = Not Applicable; LOQ = Limit of Quantitation, 0.05%

Conclusions from above data:
Formation of the USP Related Compound C was not prevented by filling Ziprasidone Injection in syringes.
As seen before, two main degradants were found that are formed under the effect of oxygen: USP Related Compound C (RRT 1.72-1.74) and a degradant with RRT 1.82.
Higher levels of the Compound C were found in this study than in the study using the vials closed under 0% oxygen. Oxygen penetration through the plunger allowed the reaction of ziprasidone with oxygen.
The development of Ziprasidone Injection-a liquid product-is feasible, provided the product is protected from oxygen.

### Example 4. Ziprasidone Injection-pH Stability

The package insert for GEODON® (ziprasidone mesylate) for Injection does not specify any pH value adjustment. The formulations prepared according to Table 1 exhibit pH values of about 3.7-3.9. Ziprasidone mesylate trihydrate is a salt of a strong acid that is solubilized by sulfobutyl ether β-cyclodextrin sodium (SBECD). The USP Monograph for Betadex Sulfobutyl Ether Sodium lists a pH value of 4.0-6.8 for a 30% (w/v) solution in carbon dioxide-free water. Therefore, it was postulated that the pH value of ziprasidone bulk solution is driven by the pH value of SBECD, and that value may not be the most favorable pH value for ziprasidone stability.

Ziprasidone and ziprasidone degradation products were determined using ultra-performance liquid chromatography (UPLC) as described in Example 6 below.

### pH Value 3.0 to 5.0

The first pH value stability study targeted a pH value range of 3.5 to 5.0. Four bulk solutions were prepared at pH values of 3.5, 3.8 (as is), 4.6, and 5.0 according to the composition shown in Table 1. Solutions were filled under nitrogen (0% oxygen) as 2.8 mL fill in 2-mL vials to minimize the headspace volume in a container closure system shown in Table 11. Some vials were also filled as 2.2 mL in 2-mL vials under atmospheric condition to investigate whether pH value would influence the previously determined oxygen sensitivity.

TABLE 9
Primary Packaging Components for Ziprasidone Injection, 20 mg/mL

| | | PART |
|---|---|---|
| COMPONENT NAME | SUPPLIER | NUMBER |
| Type I Clear Glass Tubing Vial (2 mL) | Wheaton | 223683 |
| Stopper 13 mm Serum NovaPure RP 1358 | West | 19700360 |
| 4023/50G, Flurotec ® Coated with B2-40 | Pharmaceutical | |
| | Services | |

To determine stability of the vial ingredients, the vials were placed at 40° C. and 25° C. for up to 6-months at ambient humidity in an upright position. The stability testing for the pH value of 5.0 was discontinued after 1 month due to a precipitation detected at 40° C. Similarly, testing of vials closed under 20% oxygen was discontinued after 1 month because of impurities and discoloration. Variation in pH values had no impact on ziprasidone sensitivity to oxygen.

The results for vials stored under 0% oxygen at 40° C. are shown Table 12 (pH 3.5), Table 13 (pH 3.8) and Table 14 (pH 4.6). Results for 25° C. samples can be seen in Table 15 (pH 3.5), Table 16 (pH 3.8), and Table 17 (pH 4.6). Table 18 shows a comparison of results obtained at 6 months at 25° C. and 40° C. as a function of pH.

The data presented in Tables 12-18 shows:
Similar degradation patterns were found in this study as in the other two previous studies.
The most stable pH value for Ziprasidone Injection protected from oxygen appeared to be close to pH 3.5.

TABLE 10
Stability of Ziprasidone Injection, 20 mg/mL, pH 3.5, 0% O₂ at 40° C.

| 40° C. | Specification | T = 0 | 1 Month | 2 Months | 3 Months | 6 Months |
|---|---|---|---|---|---|---|
| Pull Date | N/A | N/A | 4 Mar. 2021 | 4 Apr. 2021 | 4 May 2021 | 4 Aug. 2021 |
| Appearance | Clear, colorless to a | Clear, slightly | Clear solution | Clear solution, | Clear solution, | Clear solution, |
| | slightly pink or yellow | pink/orange solution | like “BY4” Ph | like “BY3” Ph | like “B” Ph | like “B1” Ph |
| | brownish solution free | with no visible | Eur Standard | Eur Standard | Eur Standard | Eur Standard |
| | of visible contaminants | particulates | | | | |
| Assay (HPLC), % LC | 90.0-110.0% | 101.3 | 95.9 | 97.7 | 96.6 | 98.2 |

Related Substances via HPLC (w/w %)

| | | | | | | |
|---|---|---|---|---|---|---|
| Specified-Identified | USP Related Compound C | 0.07 | 0.08 | 0.12 | 0.23 | 0.22 |
| Degradation Product | RRT 1.72-1.74 | | | | | |
| Any Unidentified | RRT 1.76-1.78 | ND | ND | ND | 0.09 | 0.17 |
| Unspecified | RRT 1.81-1.83 | 0.07 | <LOQ | 0.07 | 0.05 | 0.10 |
| Degradation Product | RRT 2.14-2.16 | ND | ND | 0.13 | 0.06 | 0.12 |
| Total Related Substances | Report Results | 0.14 | 0.17 | 0.40 | 0.56 | 0.74 |
| UV Absorbance, AU | Report Results | 0.060 | 0.160 | 0.254 | 0.313 | 0.374 |
| pH value | 2.5-6.0 | 3.60 | 3.52 | 3.77 | 3.51 | 3.57 |

| | | | | | |
|---|---|---|---|---|---|
| Test Date | 4 Feb. 2021 | 3 Mar. 2021 | 8 Apr. 2021 | 10 May 2021 | 4 Aug. 2021 |

ND = Not Detected;
LOQ = Limit of Quantitation, 0.05%

TABLE 11
Stability of Ziprasidone Injection, 20 mg/mL, pH 3.8, 0% O₂ at 40° C.

| 40° C. | Specification | T = 0 | 1 Month | 2 Months | 3 Months | 6 Months |
|---|---|---|---|---|---|---|
| Pull Date | N/A | N/A | 4 Mar. 2021 | 4 Apr. 2021 | 4 May 2021 | 4 Aug. 2021 |
| Appearance | Clear, colorless to a | Clear, slightly | Clear solution | Clear solution, | Clear solution, | Clear solution, |
| | slightly pink or yellow | pink/orange solution | like “BY4” Ph | like “BY3” Ph | like “B” Ph | like “B1” Ph |
| | brownish solution free | with no visible | Eur Standard | Eur Standard | Eur Standard | Eur Standard |
| | of visible contaminants | particulates | | | | |
| Assay (HPLC), % LC | 90.0-110.0% | 101.1 | 98.2 | 98.2 | 96.9 | 98.0 |

Related Substances via HPLC (w/w %)

| | | | | | | |
|---|---|---|---|---|---|---|
| Specified-Identified | USP Related Compound C | 0.09 | 0.12 | 0.13 | 0.20 | 0.30 |
| Degradation Product | RRT 1.72-1.74 | | | | | |
| Any Unidentified | RRT 1.76-1.78 | ND | ND | 0.06 | 0.09 | 0.26 |
| Unspecified | RRT 1.81-1.83 | 0.06 | 0.06 | 0.07 | 0.09 | 0.13 |
| Degradation Product | RRT 2.14-2.16 | ND | ND | ND | 0.08 | 0.13 |
| Total Related Substances | Report Results | 0.15 | 0.18 | 0.36 | 0.75 | 1.07 |
| UV Absorbance, AU | Report Results | 0.060 | 0.173 | 0.270 | 0.326 | 0.390 |
| pH value | 2.5-6.0 | 3.83 | 3.72 | 3.91 | 3.72 | 3.73 |

| | | | | | |
|---|---|---|---|---|---|
| Test Date | 4 Feb. 2021 | 3 Mar. 2021 | 8 Apr. 2021 | 10 May 2021 | 4 Aug. 2021 |

ND = Not Detected;
LOQ = Limit of Quantitation, 0.05%

TABLE 12
Stability of Ziprasidone Injection, 20 mg/mL, pH 4.6, 0% O₂ at 40° C.

| 40° C. | Specification | T = 0 | 1 Month | 2 Months | 3 Months | 6 Months |
|---|---|---|---|---|---|---|
| Pull Date | N/A | N/A | 4 Mar. 2021 | 4 Apr. 2021 | 4 May 2021 | 4 Aug. 2021 |
| Appearance | Clear, colorless to a | Clear, slightly | Clear solution | Clear solution, | Clear solution, | Clear solution, |
| | slightly pink or yellow | ink/orange solution | like “BY4” Ph | like “BY3” Ph | like “B” Ph | like “B2” Ph |
| | brownish solution free | with no visible | Eur Standard | Eur Standard | Eur Standard | Eur Standard |
| | of visible contaminants | particulates | | | | |
| Assay (HPLC), % LC | 90.0-110.0% | 101.0 | 98.60 | 95.9 | 94.1 | 95.1 |

Related Substances via HPLC (w/w %)

| | | | | | | |
|---|---|---|---|---|---|---|
| Specified-Identified | USP Related Compound C | 0.10 | 0.13 | 0.13 | 0.23 | 0.29 |
| Degradation Product | RRT 1.72-1.74 | | | | | |
| Any Unidentified | RRT 1.76-1.78 | ND | ND | 0.26 | 0.41 | 0.88 |
| Unspecified | RRT 1.81-1.83 | 0.05 | 0.06 | 0.07 | 0.11 | 0.14 |
| Degradation Product | RRT 2.14-2.16 | ND | ND | 0.10 | 0.08 | 0.15 |
| Total Related Substances | Report Results | 0.15 | 0.24 | 0.71 | 1.18 | 1.90 |
| UV Absorbance, AU | Report Results | 0.057 | 0.183 | 0.257 | 0.296 | 0.353 |
| pH value | 2.5-6.0 | 4.50 | 4.56 | 4.55 | 4.38 | 4.45 |

| | | | | | |
|---|---|---|---|---|---|
| Test Date | 4 Feb. 2021 | 3 Mar. 2021 | 8 Apr. 2021 | 10 May 2021 | 4 Aug. 2021 |

ND = Not Detected;
LOQ = Limit of Quantitation, 0.05%

TABLE 13
Stability of Ziprasidone Injection, 20 mg/mL, pH 3.5, 0% O₂ at 25° C.

| 25° C. | Specification | T = 0 | 3 Months | 6 Months |
|---|---|---|---|---|
| Pull Date | N/A | N/A | 4 May 2021 | 4 Aug. 2021 |
| Appearance | Clear, colorless to a slightly pink | Clear, slightly pink/ | Clear solution like | Clear solution |
| | or yellow brownish solution free | orange solution with | “D” USP Standard | like “BY3” |
| | of visible contaminants | no visible particulates | | Ph Eur Standard |
| Assay (HPLC), | 90.0-110.0% | 101.3 | 96.5 | 96.7 |
| % LC | | | | |

Related Substances via HPLC (w/w %)

| | | | | |
|---|---|---|---|---|
| Specified- | USP Related Compound C | 0.07 | 0.05 | 0.15 |
| Identified | RRT 1.72-1.74 | | | |
| Degradation | | | | |
| Product | | | | |
| Any Unidentified | RRT 1.76-1.78 | ND | ND | ND |
| Unspecified | RRT 1.81-1.83 | 0.07 | 0.12 | 0.09 |
| Degradation | RRT 2.14-2.16 | ND | ND | ND |
| Product | | | | |
| Total Related | Report Results | 0.14 | 0.22 | 0.24 |
| Substances | | | | |
| UV | Report Results | 0.060 | 0.134 | 0.176 |
| Absorbance, | | | | |
| AU | | | | |
| pH value | 2.5-6.0 | 3.60 | 3.57 | 3.57 |
| | Test Date | 4 Feb. 2021 | 10 May 2021 | 4 Aug. 2021 |

ND = Not Detected; LOQ = Limit of Quantitation, 0.05%

TABLE 14
Stability of Ziprasidone Injection, 20 mg/mL, pH 3.8, 0% O₂ at 25° C.

| 25° C. | Specification | T = 0 | 3 Months | 6 Months |
|---|---|---|---|---|
| Pull Date | N/A | N/A | 4 May 2021 | 4 Aug. 2021 |
| Appearance | Clear, colorless to a slightly pink | Clear, slightly pink/ | Clear solution like | Clear solution |
| | or yellow brownish solution free | orange solution with | “D” USP Standard | like “BY3” |
| | of visible contaminants | no visible particulates | | Ph Eur Standard |
| Assay (HPLC), | 90.0-110.0% | 101.1 | 98.6 | 99.9 |
| % LC | | | | |

Related Substances via HPLC (w/w %)

| | | | | |
|---|---|---|---|---|
| Specified- | USP Related Compound C | 0.09 | 0.20 | 0.17 |
| Identified | RRT 1.72-1.74 | | | |
| Degradation | | | | |
| Product | | | | |
| Any | RRT 1.76-1.78 | ND | 0.05 | <LOQ |
| Unidentified | RRT 1.81-1.83 | 0.06 | 0.09 | 0.09 |
| Unspecified | RRT 2.14-2.16 | ND | ND | ND |
| Degradation | | | | |
| Product | | | | |
| Total Related | Report Results | 0.15 | 0.43 | 0.31 |
| Substances | | | | |
| UV | Report Results | 0.060 | 0.141 | 0.186 |
| Absorbance, | | | | |
| AU | | | | |
| pH value | 2.5-6.0 | 3.83 | 3.75 | 3.79 |
| Test Date | | 4 Feb. 2021 | 10 May 2021 | 4 Aug. 2021 |

ND = Not Detected; NT = Not Tested; NA = Not Applicable; LOQ = Limit of Quantitation, 0.05%

TABLE 15
Stability of Ziprasidone Injection, 20 mg/mL, pH 4.6, 0% O₂ at 25° C.

| 25 ° C. | Specification | T = 0 | 3 Months | 6 Months |
|---|---|---|---|---|
| Pull Date | N/A | N/A | 4 May 2021 | 4 Aug. 2021 |
| Appearance | Clear, colorless to a slightly pink | Clear, slightly pink/ | Clear solution like | Clear solution like “BY3” |
| | or yellow brownish solution free | orange solution with | “D” USP Standard | Ph Eur Standard |
| | of visible contaminants | no visible particulates | | |
| Assay (HPLC), | 90.0-110.0% | 101.0 | 97.4 | 97.3 |
| % LC | | | | |

Related Substances via HPLC (w/w %)

| | | | | |
|---|---|---|---|---|
| Specified- | USP Related Compound C | 0.10 | 0.16 | 0.20 |
| Identified | RRT 1.72-1.74 | | | |
| Degradation | | | | |
| Product | | | | |
| Any | RRT 1.76-1.78 | ND | 0.10 | 0.17 |
| Unidentified | RRT 1.81-1.83 | 0.05 | 0.13 | 0.07 |
| Unspecified | RRT 2.14-2.16 | ND | ND | ND |
| Degradation | | | | |
| Product | | | | |
| Total Related | Report Results | 0.15 | 0.61 | 0.57 |
| Substances | | | | |
| UV | Report Results | 0.057 | 0.157 | 0.191 |
| Absorbance, | | | | |
| AU | | | | |
| pH value | 2.5-6.0 | 4.50 | 4.44 | 4.42 |
| Test Date | | 4 Feb. 2021 | 10 May 2021 | 4 Aug. 2021 |

ND = Not Detected; LOQ = Limit of Quantitation, 0.05%

TABLE 16
Effect of pH 3.5 to 4.6 - 6-Month Storage at 25° C. and 40° C. Under 0% Oxygen

| | 6 Months 0% Oxygen | | | | | |
|---|---|---|---|---|---|---|
| | 25° C. | | | 40° C. | | |
| Test | pH 3.5 | pH 3.8 | pH 4.6 | pH 3.5 | pH 3.8 | pH 4.6 |
| USP Related Compound C | 0.15 | 0.17 | 0.20 | 0.22 | 0.30 | 0.29 |
| RRT 1.72-1.74 | | | | | | |
| RRT 1.76-1.78 | ND | <LOQ | 0.17 | 0.17 | 0.26 | 0.88 |
| RRT 1.81-1.83 | 0.09 | 0.09 | 0.07 | 0.10 | 0.13 | 0.14 |
| RRT 2.14-2.16 | ND | ND | ND | 0.12 | 0.13 | 0.15 |
| RRT 2.21-2.23 | ND | ND | ND | 0.05 | 0.10 | 0.09 |
| Total Related | 0.24 | 0.31 | 0.57 | 0.74 | 1.07 | 1.90 |
| Substances, % w/v | | | | | | |
| Assay, % Label Claim | 96.7 | 99.9 | 97.3 | 98.2 | 98.0 | 95.1 |
| pH | 3.57 | 3.79 | 4.42 | 3.57 | 3.73 | 4.45 |
| UV Absorbance at 420 nm | 0.176 | 0.186 | 0.191 | 0.374 | 0.390 | 0.353 |

pH 2.8-3.5

The second pH value study extended the pH range below pH 3.5 to find the most stable pH value range. A new bulk solution was prepared according to the composition shown in Table 1, separated into three aliquots and the pH value of each aliquot was adjusted to 2.8, 3.2, and 3.5 with methansesulfonic acid, respectively. Each solution was filled again as 2.8 mL fil in 2-mL vials under nitrogen (Table 11). Vials were placed on stability at 40° C. and 25° C. for 6 months at ambient humidity in an upright position. The 6-month data for both temperatures are presented in 10 Table 19.

TABLE 17
Effect of pH 2.8 to 3.5 - 6-Month Storage at 25° C. and 40° C. Under 0% Oxygen

| | 6 Months 0% Oxygen | | | | | |
|---|---|---|---|---|---|---|
| | 25° C. | | | 40° C. | | |
| Test | pH 2.8 | pH 3.2 | pH 3.5 | pH 2.8 | pH 3.2 | pH 3.5 |
| USP Related Compound C | 0.20 | 0.18 | 0.21 | 0.18 | 0.25 | 0.26 |
| RRT 1.72-1.74 | | | | | | |
| RRT 1.76-1.78 | ND | ND | ND | 0.10 | 0.12 | 0.26 |
| RRT 1.81-1.83 | 0.10 | 0.09 | 0.09 | 0.10 | 0.12 | 0.15 |
| RRT 2.14-2.16 | ND | ND | ND | 0.09 | 0.09 | 0.08 |
| RRT 2.21-2.23 | ND | ND | 0.09 | 0.14 | 0.13 | 0.16 |
| Total Related | 0.30 | 0.27 | 0.35 | 0.66 | 0.77 | 0.84 |
| Substances, % w/v | | | | | | |
| Assay, % Label Claim | 95.90 | 97.1 | 96.8 | 98.20 | 96.1 | 95.8 |
| pH value | 2.75 | 3.13 | 3.36 | 2.71 | 3.10 | 3.37 |
| UV Absorbance at 420 nm | 0.144 | 0.160 | 0.183 | 0.360 | 0.361 | 0.376 |

Review of the data indicates that the most stable pH value is about pH 3.2. This pH value corresponds to a pH value of ziprasidone in water determined on a 1% w/v aqueous ziprasidone suspension at 25° C.

### Example 5. Ziprasidone Injection—Filled in Syringes with Secondary Packaging

As shown above, ziprasidone filled in syringes was not stable due to a penetration of oxygen through the plunger. Therefore, an additional study was initiated to demonstrate the feasibility of Ziprasidone Mesylate Injection with a pH value range of about 3.0 to about 3.5 filled in syringes and pouched with oxygen scavengers for full oxygen protection.

A new batch was prepared according to the composition shown in Table 20. Filtered bulk solution was filled in syringes and pouched subsequently into a secondary packaging containing oxygen scavenger (Table 21).

TABLE 18
Composition of Formulation for
Ziprasidone Injection 20 mg/mL, pH 3.25

| | | | Compounded |
|---|---|---|---|
| Ingredient | Function | % | Amount/mL |
| Ziprasidone Mesylate | Active | 2.47% w/v, eq to | 24.7 mg, |
| eq to ziprasidone | Ingredient | 2.0% w/v | eq to 20 mg |
| β-Cyclodextrin | Solubilizer | 29.4% w/v | 294 mg |
| sulfobutyl | | | |
| ether sodium, NF | | | |
| 0.1N Methanesulfonic | pH | Q.S. to pH 3.25 | Q.S. to |
| Acid | Adjustment | | pH 3.25 |
| Purified Water | Diluent | Q.S. to 100% v/v | Q.S. to 1 mL |

TABLE 19
Primary and Secondary Packaging Components
for Ziprasidone Injection 20 mg/mL

| Component | Supplier |
|---|---|
| Syringe EZ-Fill 1 mL Long ITC 7025/65 | Ompi |
| Plunger, Article 2340 4023/50 Gray B2-40 | West Pharmaceutical |
| | Services |
| Aluminum Mylar Pouches, 7 × 4.7 inches | QQ Studio |
| AGELESS ZPT-200MBC Oxygen Scavenger | Mitsubishi Gas |
| | America, Inc |

Syringes packaged in secondary packages with oxygen scavengers were placed on stability at 40° C. and 25° C. for 6-months at ambient humidity in a horizontal position. The study shows 0 that secondary packaging with oxygen scavengers provides greatly reduced degradation of the ziprasidone in syringes when compared to syringes not stored in secondary packaging with oxygen scavengers.

### Example 6. Ultra-Performance Liquid Chromatography (UPLC) Conditions for Ziprasidone

Diluent: methanol
Column: Phenomenex Kinetex C18 2.6 μm 150×3 mm Column, PN 00F-4462-Y0
Mobile Phase A: 0.1% TFA in Water
Mobile Phase B: 0.05% TFA in ACN
Standard: 0.6 mg/mL ziprasidone
Flow Rate: 1.0 mL/min
Run Time: 25 minutes
Column Temperature: 40° C.±3° C.
Injection Volume: 1 μL
UV Wavelength: 229 nm
Autosampler Temp.: 5° C.

### Gradient

| Time (minutes) | % Mobile Phase A | % Mobile Phase B |
|---|---|---|
| 0.00 | 90 | 10 |
| 1.00 | 90 | 10 |
| 5.00 | 72 | 28 |
| 8.00 | 69 | 31 |
| 10.00 | 69 | 31 |
| 13.00 | 64 | 36 |
| 18.00 | 50 | 50 |
| 18.10 | 5 | 95 |
| 21.00 | 5 | 95 |
| 21.10 | 90 | 10 |
| 25.00 | 90 | 10 |

Under these conditions, ziprasidone elutes at about 7.6 minutes.

### REFERENCE

PCT Patent Publication WO2020/257134 A1.

In view of the above, it will be seen that several objectives of the invention are achieved, and other advantages attained.

As various changes can be made in the above methods and compositions without departing from the scope of the invention, it is intended that all matter contained in the above description shall be interpreted as illustrative and not in a limiting sense.

All references cited in this specification, including but not limited to U.S. Patents, patent publications and non-patent literature, and references cited therein, are hereby incorporated by reference. The discussion of the references herein is intended merely to summarize the assertions made by the authors and no admission is made that any reference constitutes prior art. Applicants reserve the right to challenge the accuracy and pertinence of the cited references.

As used herein, in particular embodiments, the terms “about” or “approximately” when preceding a numerical value indicates the value plus or minus a range of 10%. Where a range of values is provided, it is understood that each intervening value, to the tenth of the unit of the lower limit unless the context clearly dictates otherwise, between the upper and lower limit of that range and any other stated or intervening value in that stated range is encompassed within the disclosure. That the upper and lower limits of these smaller ranges can independently be included in the smaller ranges is also encompassed within the disclosure, subject to any specifically excluded limit in the stated range. Where the stated range includes one or both of the limits, ranges excluding either or both of those included limits are also included in the disclosure.

The indefinite articles “a” and “an,” as used herein in the specification and in the embodiments, unless clearly indicated to the contrary, should be understood to mean “at least one.”

The phrase “and/or,” as used herein in the specification and in the embodiments, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Additional elements can optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the embodiments, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of.” or, when used in the embodiments, “consisting of,” refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.” “Consisting essentially of,” when used in the embodiments, shall have its ordinary meaning as used in the field of patent law.

As used herein in the specification and in the embodiments, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements can optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

1. An aqueous formulation that contains a psychotropic effective amount ziprasidone or a pharmaceutically acceptable salt and/or hydrate thereof, being suitable for intramuscular injection, wherein the aqueous formulation when stored at 20-25° C. for 12 months:
(a) provides greater than 98% purity of the ziprasidone, its pharmaceutically acceptable salt and/or hydrate; and
(b) has less than 0.5% of USP Related Compound C with respect to ziprasidone itself.

2. (canceled)

3. (canceled)

4. The aqueous formulation of claim 1, wherein the ziprasidone or a pharmaceutically acceptable salt and/or hydrate thereof is ziprasidone mesylate trihydrate.

5. (canceled)

6. (canceled)

7. The formulation of claim 4, further comprising β-cyclodextrin sulfobutyl ether sodium (SBECD).

8. The formulation of claim 7, wherein about 294 mg SBECD is present per 20 mg ziprasidone.

9. The formulation of claim 8, wherein the ziprasidone is present at about 20 mg/mL.

10. (canceled)

11. The formulation of claim 9, in a reduced oxygen headspace container.

12. The formulation of claim 11, wherein the reduced oxygen headspace container is a vial or ampule with N₂ gas in the headspace.

13. The formulation of claim 12, in a container with minimal oxygen headspace.

14. The formulation of claim 13, wherein the container is a syringe.

15. The formulation of claim 13, having a volume of about 0.5 to about 1.5 mL and contains about of about 20 mg of ziprasidone per mL.

16. The formulation of claim 15, having a pH value of about 2.8 to about 4.0.

17. The formulation of claim 16, in a syringe within secondary packaging that prevents penetration of oxygen through a plunger of the syringe.

18. The formulation of claim 17, wherein the secondary packaging is a pouch that comprises an oxygen scavenger.

19. (canceled)

20. (canceled)
